(19) United States
(12) Patent Application Publication
(10) Pub. No.: US 2011/0264099 A1
(43) Pub. Date: Oct. 27, 2011

(54) MULTI-DIRECTIONAL CEMENT DELIVERY SYSTEM

(75) Inventor: Mark B. Quinto, San Jose, CA (US)

(73) Assignee: KYPHON SÀRL, Neuchatel (CH)

(21) Appl. No.: 12/766,763

(22) Filed: Apr. 23, 2010

(51) Int. Cl. A61B 17/56

(52) U.S. Cl. 606/94

(57) ABSTRACT

An instrument for performing a medical procedure includes a nozzle sized to fit through a cannula, and a plunger sized to be advanced through the nozzle and urge material through a distal tip of the nozzle. The plunger includes an interior lumen and a distal tip having a side port, such that after material delivery from the nozzle, the distal tip of the plunger can be extended beyond the distal tip of the nozzle, and additional material delivery can be performed via the side port of the plunger. This dual action material delivery capability can beneficially enhance material placement at the target location, and can also enable delivery of different materials to the target location without having to exchange delivery tools.

### FIELD OF THE INVENTION

The invention relates to a system and method for performing a surgical procedure, and in particular, to a system for efficiently delivering bone filler material.

### BACKGROUND OF THE INVENTION

For many individuals in our aging world population, undiagnosed and/or untreatable bone strength losses have weakened these individuals' bones to a point that even normal daily activities pose a significant threat of fracture. In one common scenario, when the bones of the spine are sufficiently weakened, the compressive forces in the spine can cause fracture and/or deformation of the vertebral bodies. For sufficiently weakened bone, even normal daily activities like walking down steps or carrying groceries can cause a collapse of one or more spinal bones. A fracture of the vertebral body in this manner is typically referred to as a vertebral compression fracture. Other commonly occurring fractures resulting from weakened bones can include hip, wrist, knee and ankle fractures, to name a few.

Fractures such as vertebral compression fractures often result in episodes of pain that are chronic and intense. Aside from the pain caused by the fracture itself, the involvement of the spinal column can result in pinched and/or damaged nerves, causing paralysis, loss of function, and intense pain which radiates throughout the patient's body. Even where nerves are not affected, however, the intense pain associated with all types of fractures is debilitating, resulting in a great deal of stress, impaired mobility and other long-term consequences. For example, progressive spinal fractures can, over time, cause serious deformation of the spine (“kyphosis”), giving an individual a hunched-back appearance, and can also result in significantly reduced lung capacity and increased mortality.

Until recently, treatment options for vertebral compression fractures, as well as other serious fractures and/or losses in bone strength, were extremely limited—mainly pain management with strong oral or intravenous medications, reduced activity, bracing and/or radiation therapy, all with mediocre results. Because patients with these problems are typically older, and often suffer from various other significant health complications, many of these individuals are unable to tolerate invasive surgery. In addition, to curb further loss of bone strength, many patients are given hormones and/or vitamin/mineral supplements—again with mediocre results and often with significant side effects.

In an effort to more effectively and directly treat vertebral compression fractures, minimally invasive techniques such as vertebroplasty and, subsequently, kyphoplasty, have been developed. Both techniques involves the percutaneous injection of a flowable reinforcing material, usually polymethylmethacrylate (PMMA—commonly known as bone cement), into a fractured, weakened, or diseased vertebral body. Shortly after injection, the liquid filling material hardens or polymerizes, desirably supporting the vertebral body internally, alleviating pain and preventing further collapse of the injected vertebral body.

In a vertebroplasty procedure, a needle is inserted directly into a vertebral body, and the bone cement is dispensed from the needle. Because the liquid bone cement naturally follows the path of least resistance within bone, and because the small-diameter needles used to deliver bone cement in vertebroplasty procedure typically require either high delivery pressures to ensure that the bone cement remains within the already compromised vertebral body is a significant concern in vertebroplasty procedures.

Kyphoplasty addresses this issue by first creating a cavity within the vertebral body (e.g., with an inflatable balloon to enable the procedure to be performed percutaneously) and then filling that cavity with bone filler material. The cavity provides a natural containment region that minimizes the risk of bone filler material escape from the vertebral body. An additional benefit of kyphoplasty is that the creation of the cavity can also restore the original height of the vertebral body, further enhancing the benefit of the procedure.

The actual filling of the cavity within the vertebral body is typically performed using an elongate nozzle that is guided through the same percutaneous path (cannula) through which the cavity creation device had been deployed. The nozzle either delivers the bone filler material through an open tip (i.e., in a direction substantially aligned with the longitudinal axis of the nozzle, or through a side port (i.e., in a direction substantially non-aligned with the longitudinal axis of the nozzle). Depending on the bone filler material delivery characteristic desired by the user, a nozzle with either an open tip or a side port can be selected. However, in certain instances, it can be desirable to provide a bone filler material delivery device that provides both non-directional (i.e., “straight-through” material delivery via an open tip) and directional (i.e., non-aligned material delivery through a side port) delivery capabilities.

### SUMMARY OF THE INVENTION

By providing a material delivery nozzle sized to receive a cannulated plunger having a side port, a percutaneous surgical material delivery system can provide both non-directional and directional material delivery capabilities.

In one embodiment, a surgical material delivery system includes a nozzle sized to fit through a cannula, and a plunger sized to be advanced through the nozzle and urge material in the nozzle out the distal tip of the nozzle. The plunger exhibits a closed tip, but also includes an inner lumen and a side port(s) to enable additional material delivery after plunging the material from the nozzle. Consequently, the surgical material system can enable efficient and effective material delivery to a target surgical location.

In one embodiment, the material delivery system can further include a secondary plunger sized to be advanced through the plunger and urge material in the plunger out the side port of the plunger. In various other embodiments, the material delivery system can further include a syringe or hydraulic delivery system for forcing material through the plunger and out the side port. Such systems would enable material delivery in quantities greater than just the volume of the inner lumen of the plunger. In addition, a syringe, pump, or hydraulic pressure generator could be controlled remotely from the material delivery system (e.g., via flexible tubing) to allow the user to remain outside any radiation field in the vicinity of the target location (e.g., a fluoroscopic x-ray field).

In another embodiment, a material delivery system that includes a nozzle and cannulated plunger can be incorporated into a kit for a surgical procedure that includes a cannula, wherein the nozzle is sized to be placed in the cannula for access to the target location. In some embodiments, such a kit can further include additional instruments for the procedures (e.g., access tools such as obturators and/or drills, bone manipulation devices such as curettes and/or inflatable bone tamps for kyphoplasty procedures, etc.) and/or instructions for using the material delivery system.

In various other embodiments a surgical or instructional procedure can be performed using a material delivery system that includes a nozzle and cannulated plunger. A percutaneous path can be created to a target location (e.g., using a cannula or even just inserting the (optionally sharpened) nozzle directly into the target location), after which material (e.g., bone filler material) is dispensed into the target location from the distal tip of the nozzle in response to advancement of the plunger. The side port of the plunger is then exposed beyond the distal tip of the nozzle and additional material (not necessarily the same material dispensed from the nozzle) is dispensed from the plunger through the side port. In some embodiments, the plunger can be rotated and/or moved longitudinally during this dispensing operation to provide the desired delivery characteristics.

As will be realized by those of skilled in the art, many different embodiments of a surgical instrument, kit, and/or methods of using a surgical instrument incorporating directional and non-directional material delivery capabilities are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIGS. 1A-1F are diagrams of a material delivery system that includes a material delivery nozzle and a cannulated plunger.

FIG. 2 is a diagram of an exemplary surgical kit that includes the material delivery system of FIGS. 1A-1F.

FIGS. 3A-3H show an exemplary surgical procedure using the material delivery system of FIGS. 1A-1F.

FIGS. 4A-4B show another exemplary surgical procedure using the material delivery system of FIGS. 1A-1F.

FIG. 5 is a flow diagram for the use of the material delivery system of FIGS. 1A-1F.

### DETAILED DESCRIPTION

By providing a material delivery nozzle sized to receive a cannulated plunger having a side port, a percutaneous surgical material delivery system can provide both non-directional and directional material delivery capabilities. The actual surgical material delivered can be any material that can be used in a surgical procedure, such as bone filler material (e.g., PMMA), granulized or pelletized material such as bone morphongenic protein (BMP), graft material, or even solid materials that can be forced from the material delivery nozzle and/or the cannulated plunger.

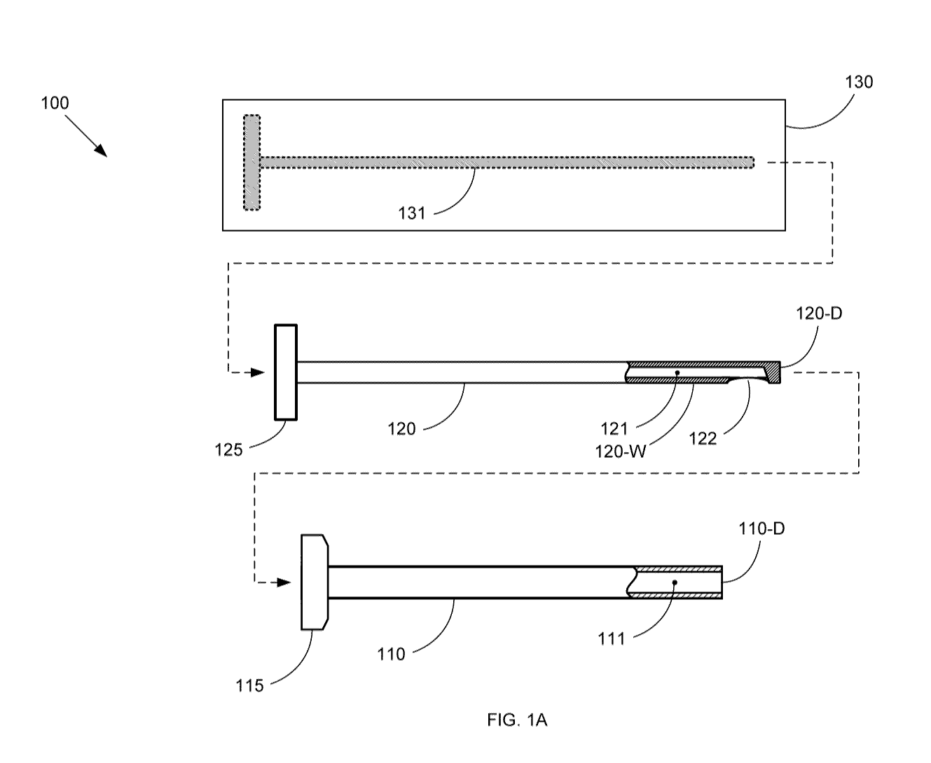

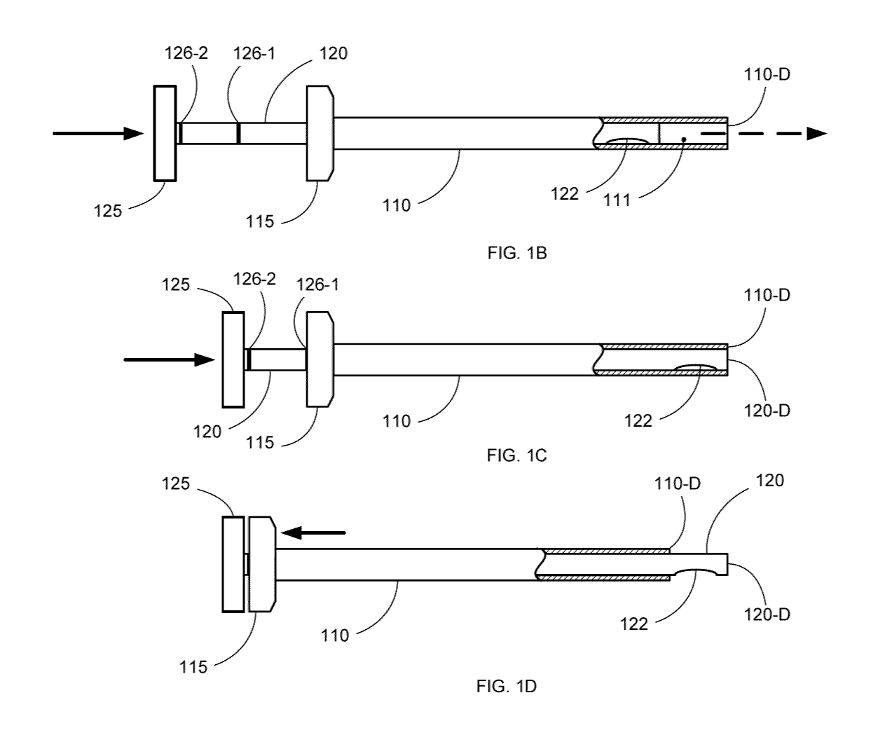

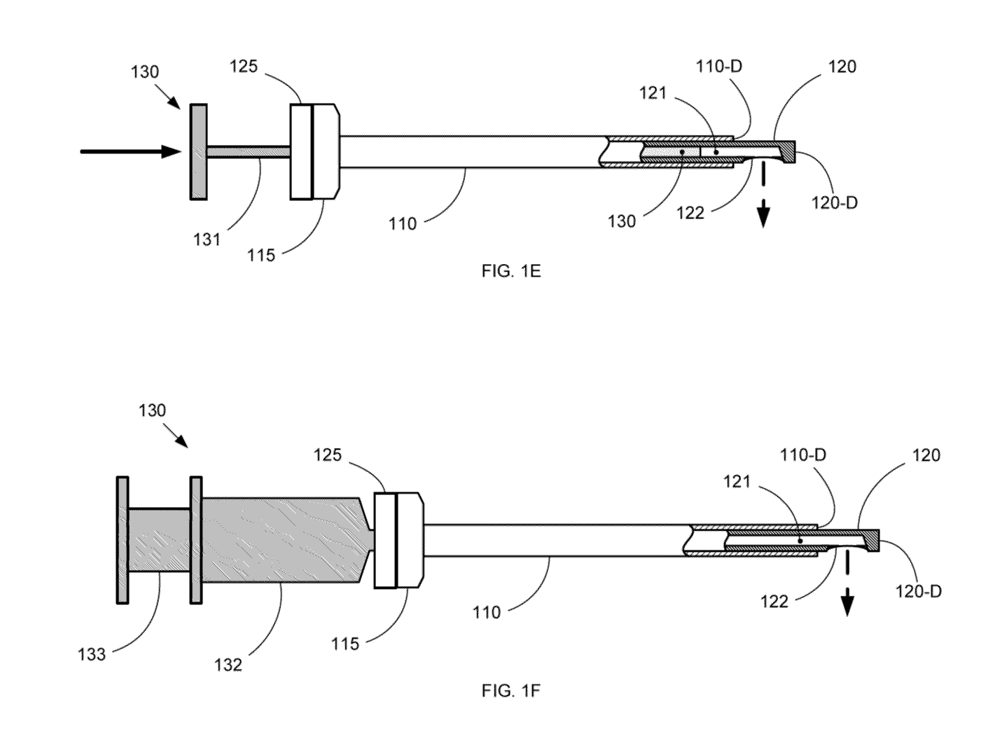

FIG. 1A shows an exemplary surgical material delivery system 100 that includes a nozzle 110 and a plunger 120. Nozzle 110 is an elongate element defining an interior nozzle lumen 111, that runs through nozzle 110. An optional handle 115 towards a proximal end of nozzle 110 can provide a gripping/manipulating location. Note that the particular location, shape, and configuration of handle 115 shown in FIG. 1A is for exemplary purposes only, and in various other embodiments, handle 115 can be curved, circular, square, or any other shape, and can be located anywhere along nozzle 110.

Plunger 120 is an elongate element sized to fit within nozzle lumen 111 and express any material within nozzle lumen 111 out distal end 110-D of nozzle 110. Like nozzle 110, plunger 120 can include an optional handle 125 for gripping/manipulation that can take any size, shape and position along plunger 120. In addition, plunger 120 itself defines an inner plunger lumen 121 that can be used for material delivery. A distal end 120-D of plunger 120 is closed (solid) to enable plunger 120 to push material from within nozzle 110 without allowing that material to enter plunger lumen 121. To enable material delivery from inner plunger lumen 121, sidewall 120-W of plunger 120 includes a side port (opening) 122 that provides a passageway to plunger lumen 121. Note that while a single side port 122 is depicted for exemplary purposes, in various other embodiments, plunger 120 can include multiple side ports (e.g., ports on opposite sides of plunger 120 or any number of ports in any arrangement proximal of distal end 120-D).

As described in greater detail below, plunger 120 can be used to urge material from within nozzle 110 into a target location, and can then be used to deliver additional material from within plunger lumen 121 to the target location. This additional material delivery can be effected by a secondary material delivery element 130, as further shown in FIG. 1A. Secondary material delivery element 130 can comprise any device for causing material to flow through plunger lumen 121 and out side port 122. For example, secondary material delivery element 130 can comprise a plunger 131 sized to fit within plunger lumen 121. However, in various other embodiments, secondary material delivery element 130 can be a syringe, hydraulic pump, piston, or any other material driving mechanism or system.

FIGS. 1B-1E depict an exemplary interaction between the elements of surgical material delivery system 100. In FIG. 1B, plunger 120 is shown positioned within nozzle lumen 111. By moving plunger 120 further into nozzle 110 (i.e., in the direction of the solid arrow), material within nozzle lumen 111 is urged from the open distal end 110-D of nozzle 110 (i.e., in the direction of the dashed arrow). Note that nozzle 110 effectively doses off side port 122 of plunger 120 to prevent any material flow from within plunger 120 out through distal end 110-D of nozzle 110.

Note that in various embodiments, plunger 120 and/or nozzle 110 can include optional positional indicators 126 that provide an indication to the user of the relative positions of the flow outlets of plunger 120 and nozzle 110 (i.e., distal end 110-D and side port 122, respectively). For example, in the embodiment shown in FIG. 1B, plunger 120 includes positional indicators 126-1 and 126-2. In various embodiments, indicators 126 can be markings, features, color changes, or any other positional reference. The position of indicators 126-1 and 126-2 can then be used to determine the relationship between the distal ends of plunger 120 and nozzle 110.

For example, in FIG. 1C, as plunger 120 is further inserted into nozzle 110, the distal end 120-D of plunger 120 eventually aligns with the distal end 110-D of nozzle 110. At the same time, indicator 126-1 aligns with handle 115 of nozzle 110. Therefore, even if the distal end 110-D of nozzle 110 is hidden from view (e.g., by a cannula, as would be typical during a percutaneous surgical procedure), this tip alignment between plunger 120 and nozzle 110 can be determined by the relationship between indicator 126-1 and handle 125. Note that in various other embodiments, indicator 126-1 can be examined relative to any other feature of nozzle 110 (e.g., the proximal end of nozzle 110 or a slot/window in nozzle 110).

The alignment of distal tip 120-D of plunger 120 with distal tip 110-D of nozzle 110 means that all the material formerly within nozzle lumen 111 has been expressed from nozzle 110. At this point, plunger 120 can be further advanced through nozzle 110 as shown in FIG. 10 such that distal tip 120-D of plunger 120 extends out from distal tip 110-D of nozzle 110, thereby exposing side port 122. Note that while this advancement of plunger 120 through nozzle 110 can be achieved via moving plunger 120 distally further into nozzle 110, in various other embodiments, nozzle 110 can be moved proximally towards plunger 120 (as indicated by the solid arrow), or by a combination of both nozzle 110 and plunger 120 motion. Note further that in some embodiments, plunger 120 can include positional indicator 126-2 that indicates when side port 122 is fully extended beyond distal tip 110-D of nozzle 110. In various other embodiments, this “exposed state” for side port 122 can be indicated by physical means (e.g., when handle 115 (or any other feature) of nozzle 110 abuts handle 125 (or any other feature) of plunger 120).

Then, as shown in FIG. 1E, secondary material delivery element 130 can be used to express material from within plunger lumen 121 out through side port 122 (as indicated by the dashed arrow). For exemplary purposes, secondary material delivery element 130 is depicted as a simple plunger 130 sized to fit within plunger lumen 121 and directly urge material from plunger 120 (as indicated by the solid arrow). However, as noted above, secondary material delivery element 130 can be any mechanism for driving material through plunger lumen 121 and out though side port 122.

For example, in another embodiment, secondary material delivery element 130 can be a syringe having a barrel 132 releasably coupled to plunger 120 (e.g., via a threaded connection, a press or wedge fit, a snap fit, or any other connection), as shown in FIG. 1F. Barrel 132 can be filled with the desired delivery material and then connected to plunger 120. A plunger 133 can then be used to express material from within barrel 132 into plunger lumen 121 and out through side port 122. In various other embodiments, syringe 130 could be coupled to plunger 120 via a flexible tubing, or syringe 130 could itself be driven by a remote actuator (e.g., via hydraulic lines or cables).

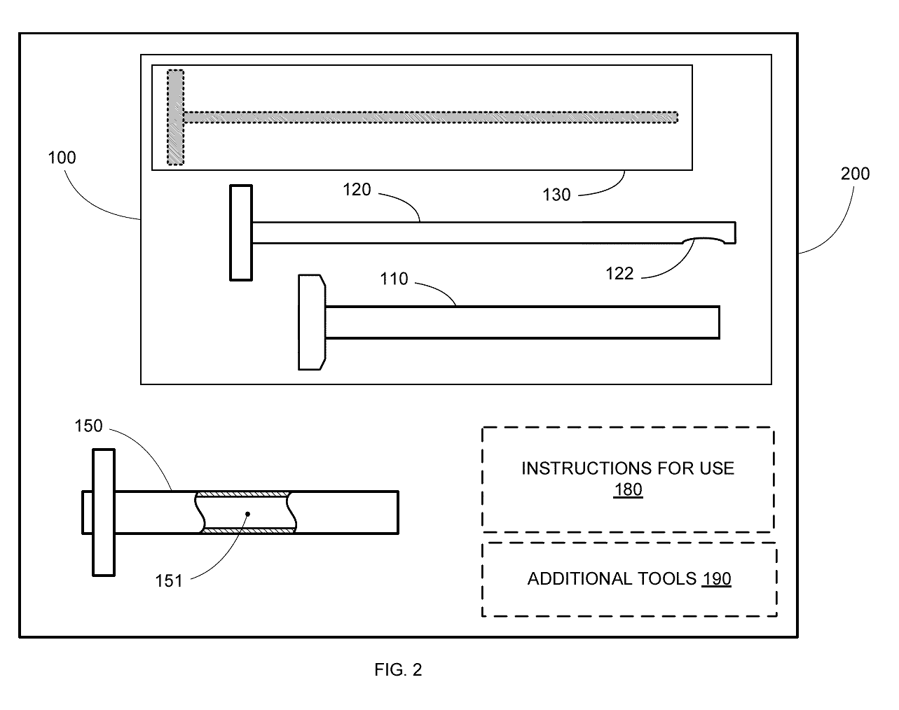

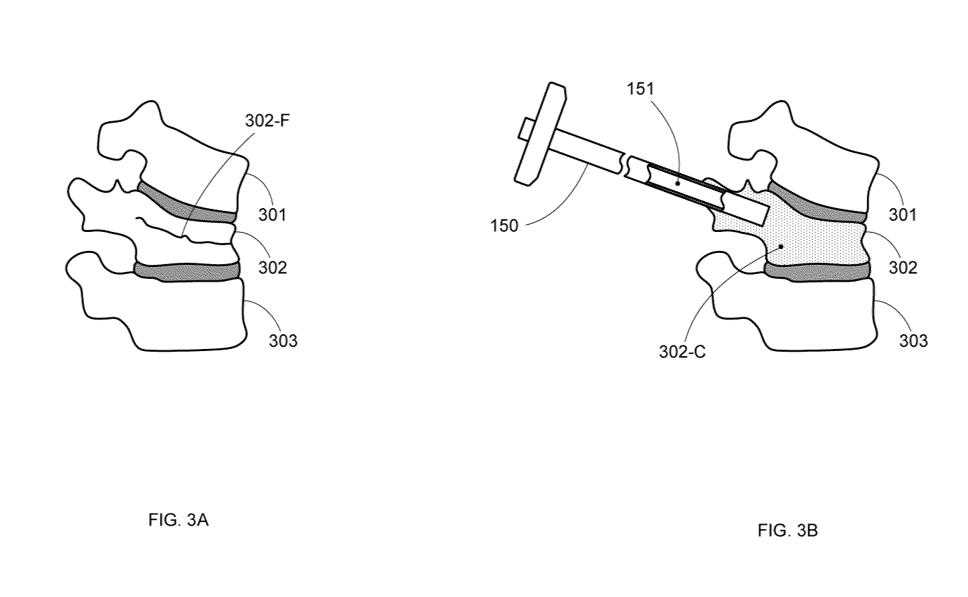

FIG. 2 shows a diagram of a kit 200 for use in performing a surgical procedure. Kit 200 includes surgical material delivery system 100 (as described with respect to FIGS. 1A-1F) that includes nozzle 110, cannulated plunger 120, and secondary material delivery element 130. Kit 200 further includes a cannula 150 that defines an interior cannula lumen 151 for providing a percutaneous access path to a target surgical location. Nozzle 110 is sized to fit through cannula lumen 151 to enable material delivery to the target surgical location. Kit 200 can further include optional additional instruments 180 (e.g., introducer tools, cavity creation tools, and/or any other useful instruments for the desired surgical procedure) and optional directions for use 190 that provide instructions for using material delivery system 100 and optional additional instruments 190 (e.g., instructions for performing a vertebroplasty or kyphoplasty procedure using material delivery system 100 and optional additional instruments 180).

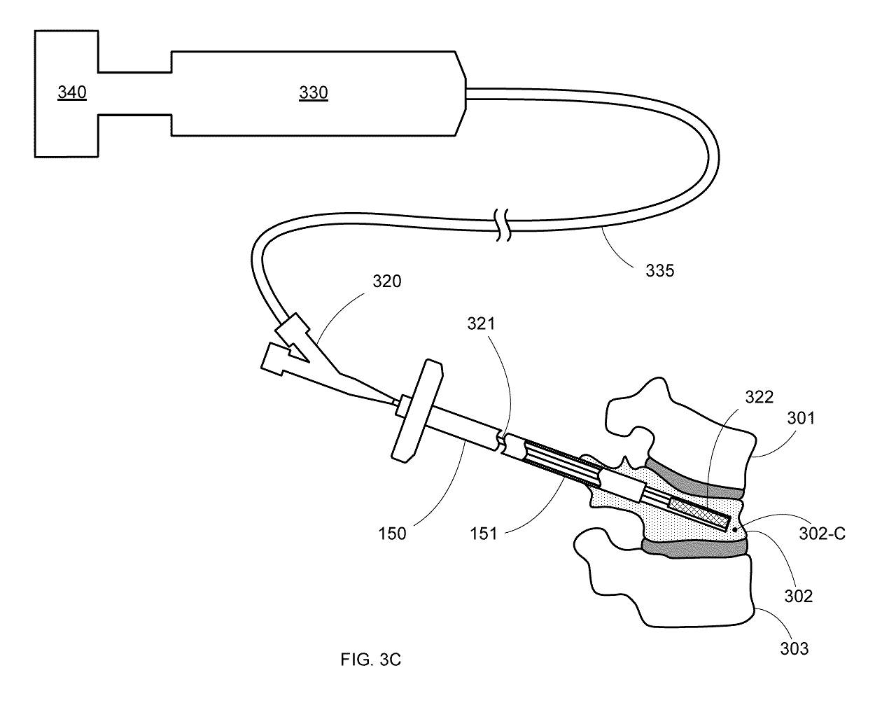

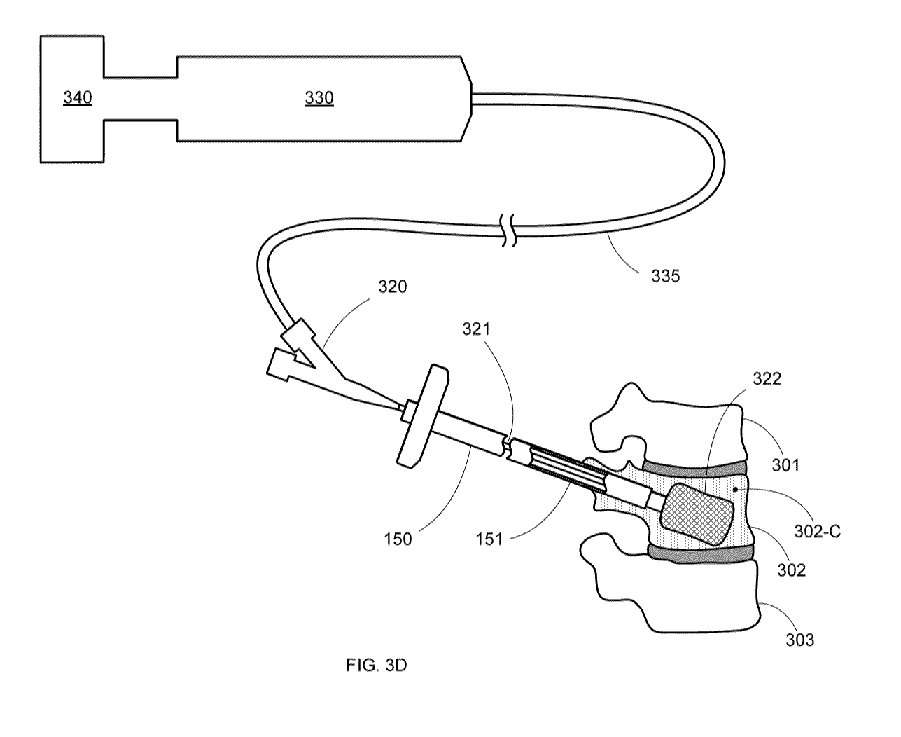

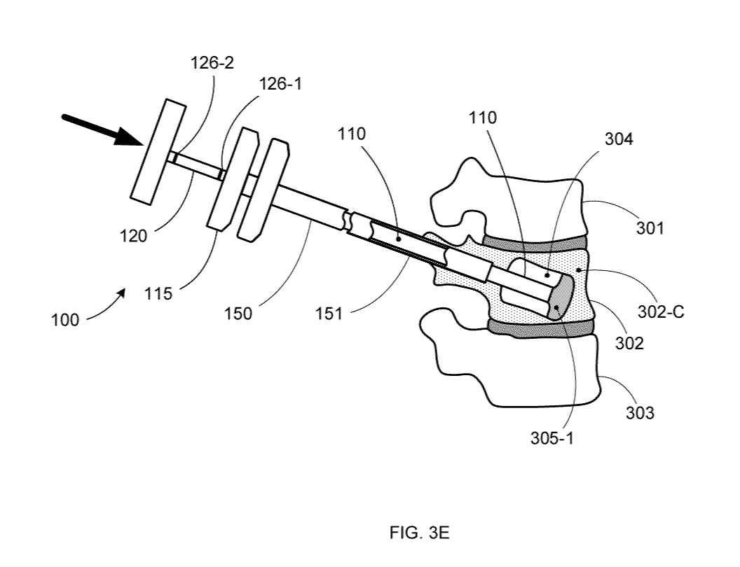

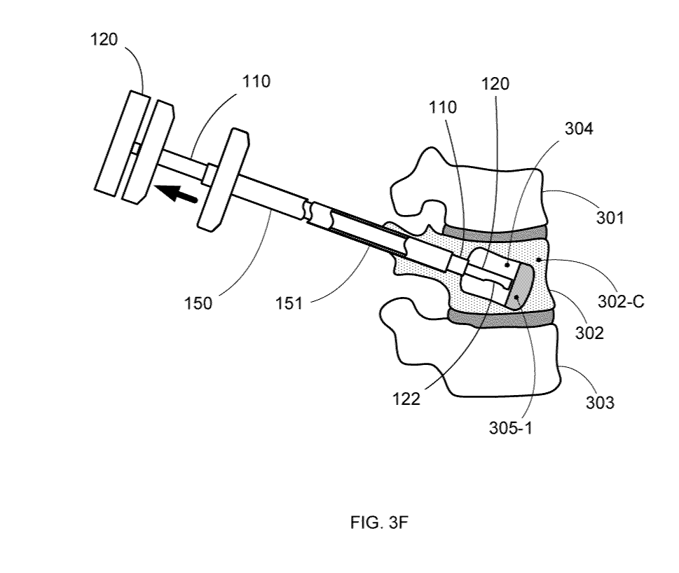

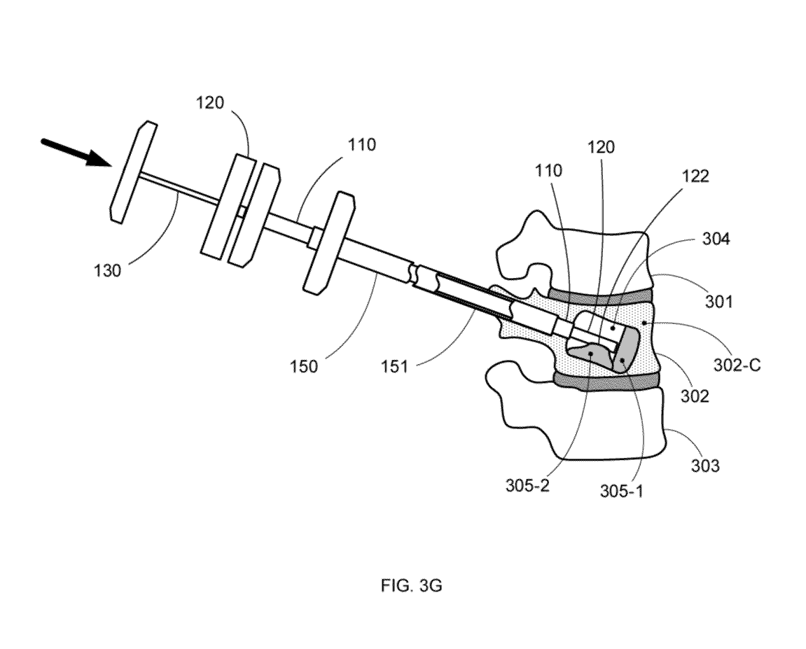

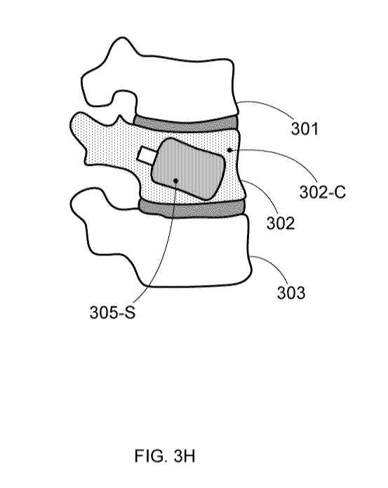

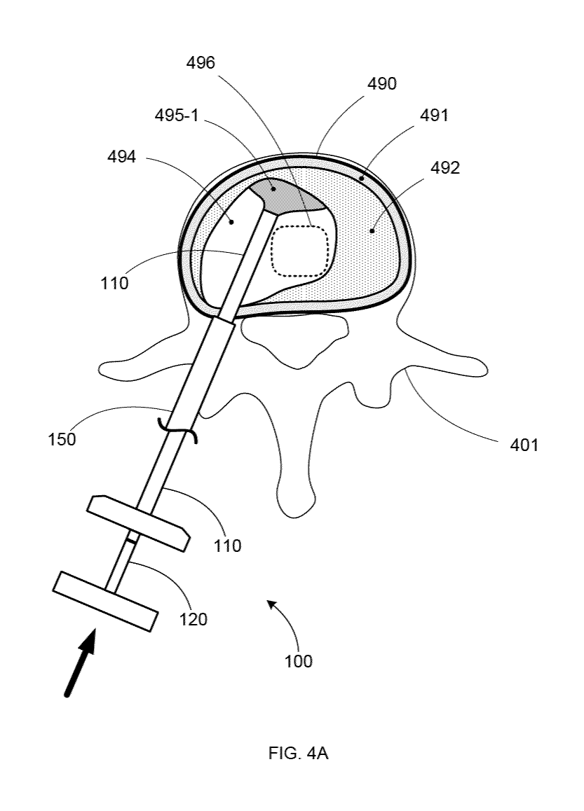

FIGS. 3A-3H show an exemplary kyphoplasty procedure using surgical material delivery system 100. FIG. 3A shows a portion of a human vertebral column having vertebrae 301, 302, and 303. Note that while human vertebrae are depicted for exemplary purposes, in various other embodiments, surgical material delivery system 100 can be used in other human bones (e.g., long bones or joints), animal bones, or even artificial bones (e.g., use in sawbones for training purposes).

As depicted in FIG. 3A, vertebra 302 has collapsed due to a vertebral compression fracture (VCF) 302-F that could be the result of osteoporosis, cancer-related weakening of the bone, and/or physical trauma. VCF 302-F can create an abnormal spinal curvature that can, in turn, lead to severe pain and further fracturing of adjacent vertebral bodies.

FIG. 3B shows a cannula 150 being positioned next to the target surgical location, which in this case is the cancellous bone structure 302-C within fractured vertebra 302. In this manner, a percutaneous path to vertebra 302 is provided via interior lumen 151 of cannula 150. Typically, cannula 150 is docked into the exterior wall of the vertebral body (using either a transpedicular or extrapedicular approach) using a guide needle and/or dissector, after which a drill or other access tool (not shown) is used to create a path further into the cancellous bone 302-C of vertebra 302. However, any other method of cannula placement can be used to position cannula 150.

Then in FIG. 3C, an inflatable bone tamp 320 is placed into cannula 150. Inflatable bone tamp 320 includes a shaft 321 (e.g., a catheter), and an expandable structure 322 (e.g., a balloon or bag) at the distal end of shaft 321. Inflatable bone tamp 320 is coupled to inflation syringe 330 by a flow channel 335 (e.g., flexible tubing). For exemplary purposes, inflation syringe 330 is depicted as a syringe having a plunger 340 for expressing inflation fluid (e.g., saline solution, air, contrast solution, or any other fluid) from inflation syringe 330, through flow channel 335, through shaft 321 of inflatable bone tamp 320, and into expandable structure 322.

Note that in various other embodiments, inflation syringe 330 can be any system for delivering inflation fluid, such as a syringe, pump, or compressed gas system, among others. Furthermore, in various other embodiments, inflation syringe 330 can be directly connected to inflatable bone tamp 320. Note further that while inflatable bone tamp 320 is depicted and described as the cavity creation instrument for the instant procedure for exemplary purposes, in various other embodiments, other types of mechanical void creation devices can be used (e.g., curettes, whisks, osteotomes, jacks, rasps, drills, and/or ablation systems, among others) in place of, or in conjunction with, inflatable bone tamp 320.

Next, as shown in FIG. 3D, inflation syringe 330 is actuated to drive inflation fluid into expandable structure 322, causing expandable structure 322 to expand within fractured vertebra 302. This expansion of expandable structure 322 compresses the surrounding cancellous bone 302-C to create a cavity within vertebra 302. In addition, the lifting force generated by expandable structure 322 can beneficially restore the height of vertebra 320 to its pre-fracture state.

Once expandable structure 322 has been expanded to a desired volume and/or a desired height restoration has been achieved in vertebra 302, expandable structure 322 is deflated, and inflatable bone tamp 320 is removed from vertebra 302 (through cannula 150). Cavity 304 can then be filled with a first portion of bone filler material 305-1 (e.g., PMMA) using material delivery system 100, as shown in FIG. 3E.

Nozzle 110 is inserted through cannula 150 and into cavity 304, and plunger 120 is inserted into nozzle 110. Advancing plunger 120 (i.e., moving plunger 120 into nozzle 110) expresses bone filler material 305-1 from nozzle 110 into cavity 304 as shown in FIG. 3E. This non-directional (i.e., straight through) delivery of bone filler material 305-1 can beneficially ensure proper anterior filling of cavity 304.

Once the first portion of bone filler material 305-1 is fully expressed from nozzle 110, advancement of plunger 120 can be stopped. As described above with respect to FIGS. 1B-1D, plunger 120 can include positional indicators 126 that allow the user to monitor the position of plunger 120 within nozzle 110. Therefore, in one embodiment, advancement of plunger 120 can be stopped when positional indicator 126-1 is appropriately aligned with cannula 150 (for example, aligned with the proximal end of handle 115), indicating that the distal tip of plunger 120 is at the distal tip of nozzle 110. In other embodiments, advancement of plunger 120 can continue until at least a portion of plunger 120 extends beyond the distal tip of nozzle 110.

Nozzle 110 can then be retracted as shown in FIG. 3F to expose side port 122 of plunger 120. Note that while retraction of nozzle 110 (i.e., moving nozzle 110 away from the target surgical location—in this case cavity 304) can often be the way to reveal side port 122, as noted above, side port 122 can also be exposed by advancing plunger 120 further into nozzle 110, or by a combination of nozzle retraction and plunger advancement.

Then, secondary material delivery element 130 is used to urge a second portion of bone filler material 305-2 into cavity 304, as shown in FIG. 3G. Bone filler material 305-2 may or may not be the same material as bone filler material 305-1. Due to the directional material delivery provided by side port 122, the second portion of bone filler material 305-2 can be effectively delivered to the entire volume of cavity 304. In various embodiments, plunger 120 can be rotated and/or moved longitudinally during this fill process to ensure desired material delivery characteristics.

Once the filling operation is complete, nozzle 110, plunger 120, and cannula 150 are removed from vertebra 302 (and the patient's body) as shown in FIG. 3H. Note that because side port 122 is effectively “closed off” once plunger 120 is retracted into nozzle 110, the possibility of cement extravasation out through the path created by cannula 150 is minimized during removal of these instruments. Then, upon hardening, bone filler material portions 305-1 and 305-2 form a mass of stabilized bone filler material 305-S that provides structural support for vertebra 302, thereby substantially restoring the structural integrity of the bone and the proper musculoskeletal alignment of the spine. In this manner, the pain and attendant side effects of a vertebral compression fracture can be addressed by a minimally invasive kyphoplasty procedure.

Note that while the use of surgical material delivery system 100 is described above with respect to a kyphoplasty procedure for exemplary purposes, surgical material delivery system 100 can be used in any procedure for which controlled directional material delivery is desirable. For example, to relieve pain due to spinal disc degeneration or herniation, some or all of the disc nucleus material can be removed and replaced with stabilizing material (e.g., bone graft or cement) and mechanical support structure(s) (e.g., implants or plates/rods). The removal of nucleus material is referred to as a discectomy, and the subsequent mechanical stabilization is referred to as spinal fusion.

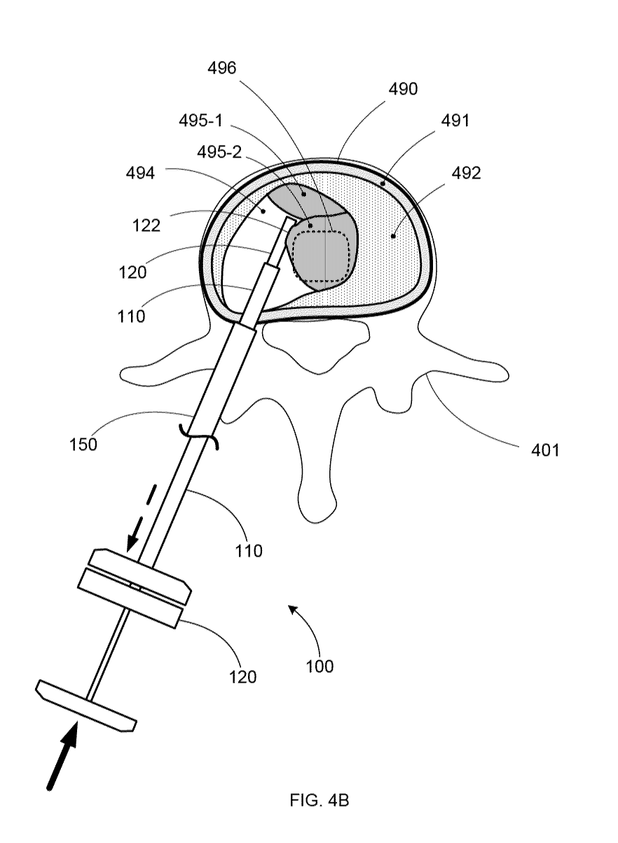

FIG. 4A shows an exemplary material delivery procedure to a disc 490. Disc 490 includes an annulus (outer wall) 491, and an interior nucleus 492. Disc 490 is located between adjacent vertebral bodies (exemplary vertebra 401 is shown for explanatory purposes), and a discectomy has been previously performed, leaving a cavity 494 in nucleus 492. An optional implant 496 (e.g., fusion cage) is positioned within cavity 494 to provide supplemental structural support.

Delivery nozzle 110 is positioned within cavity 494 through an access cannula 150, and plunger 120 is used to express a first quantity of filler material 495-1 (e.g., allograft, autograft, or bone graft substitute) from nozzle 110 into cavity 494 (as indicated by the arrow). Then, as shown in FIG. 4B, nozzle 110 is retracted (as indicated by the dashed arrow) to expose the tip of plunger 120, and a second quantity of filler material 495-2 is delivered into cavity 494 from the side port 122 of plunger 120 (in response to secondary material delivery element 130, as indicated by the solid arrow).

As noted above, plunger 120 can be rotated and/or translated along its longitudinal axis during material delivery from side port 122 to ensure proper filling of cavity 494. Once a sufficient quantity of filler material 495 has been delivered to cavity 494, material delivery system 110 and cannula 150 can be removed from disc 490, and any subsequent fusion procedure steps can be performed (e.g., attachment of rods or plates to the adjacent vertebral bodies).

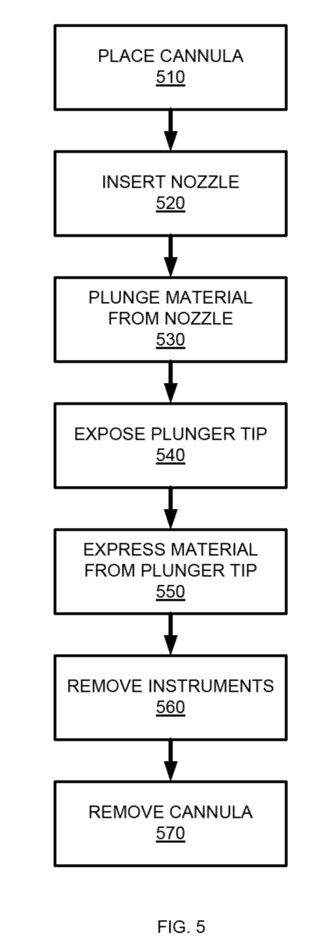

FIG. 5 shows a flow diagram of the mechanical operation described above with respect to FIGS. 3A-3H and 4A-48. In a PLACE CANNULA step 510, a cannula (e.g., cannula 150) is positioned to provide a working path to the target surgical location (e.g., human bone/disc, animal bone/disc, or cadaver/artificial bone/disc), as described with respect to FIGS. 3B and 4A. In an INSERT NOZZLE step 520, a surgical material delivery nozzle (e.g., nozzle 110) is placed into the cannula, as described with respect to FIGS. 3E and 4A. In some embodiments, this insertion operation can be preceded by various other procedures (e.g., cavity creation in cancellous bone, as described with respect to FIGS. 3C and 3D, or discectomy to create a cavity in a spinal disc).

Then, in a PLUNGE MATERIAL FROM NOZZLE step 530, a plunger (e.g., plunger 120) is used to express surgical material (e.g., bone filler material or graft) from the nozzle into the target location, as described with respect to FIGS. 3E and 4A. Once the nozzle has been emptied, the side port(s) (e.g., side port 122) of the plunger is exposed in an EXPOSE PLUNGER TIP step 540, as described with respect to FIGS. 3F and 4B. Additional surgical material is then delivered from the side port(s) to the target location in an EXPRESS MATERIAL FROM PLUNGER TIP step 550, as described with respect to FIGS. 3G and 4B. Once this filling operation is complete, the nozzle and plunger are removed from the cannula in a REMOVE INSTRUMENTS step 560, and the cannula is removed in a REMOVE CANNULA step 570, as described with respect to FIG. 3H.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

1. A surgical apparatus comprising:
a nozzle including a distal tip, the nozzle defining an interior nozzle lumen extending through the distal tip; and
a plunger sized to be advanced through the interior nozzle lumen and urge a first material from the nozzle, the plunger comprising
a sidewall defining an interior plunger lumen; and
a closed distal tip,
wherein the sidewall further defines a first opening to the interior plunger lumen proximal to the closed distal tip.

2. The surgical apparatus of claim 1, wherein the sidewall further defines a second opening to the interior plunger lumen proximal to the closed distal tip.

3. The surgical apparatus of claim 1, further comprising a secondary material delivery element for urging a second material from the plunger through the first opening.

4. The surgical apparatus of claim 3, wherein the secondary material delivery element comprises a secondary plunger sized to be advanced through the interior plunger lumen.

5. The surgical apparatus of claim 3, wherein the secondary material delivery element comprises a syringe having a tip that is releasably connectable to the plunger.

6. The surgical apparatus of claim 3, wherein the secondary material delivery element comprises:
an actuator; and
a flexible tube for coupling the actuator to the plunger,
wherein the actuator supplies pressure via the flexible tube to the interior plunger lumen to urge the second material from the plunger.

7. The surgical apparatus of claim 1, wherein the plunger includes at least one marking to visually gauge the advancement of the plunger relative to the distal tip of the nozzle, and
wherein the at least one marking indicates when the closed distal tip of the plunger is aligned with the distal tip of the nozzle.

8. The surgical apparatus of claim 1, wherein the plunger includes at least one marking to visually gauge the advancement of the plunger relative to the distal tip of the nozzle, and
wherein the at least one marking indicates when the first opening is positioned distally of the distal tip of the nozzle.

9. A kit comprising:
a cannula for establishing a path to a target location; and
a material delivery apparatus comprising:
a nozzle including a distal tip, the nozzle defining an interior nozzle lumen extending through the distal tip, the nozzle being sized to access the target location through the cannula; and
a plunger sized to be advanced through the interior nozzle lumen and urge a first material from the nozzle, the plunger comprising
a sidewall defining an interior plunger lumen; and
a closed distal tip,
wherein the sidewall further defines a first opening to the interior plunger lumen proximal to the closed distal tip.

10. The kit of claim 9, further comprising a secondary material delivery element for urging a second material from the plunger through the first opening.

11. The kit of claim 10, wherein the secondary material delivery element comprises a secondary plunger sized to be advanced through the interior plunger lumen.

12. The kit of claim 10, wherein the secondary material delivery element comprises a syringe having a tip that is releasably connectable to the plunger.

13. The kit of claim 9, wherein the target location comprises cancellous bone,
the kit further comprising an instrument for creating a cavity in the cancellous bone, the instrument being sized to access the target location through the cannula.

14. A method comprising:
creating a percutaneous path to a target surgical location;
placing a nozzle in the percutaneous path;
advancing a plunger through the nozzle to urge material in the nozzle out a distal tip of the nozzle and into the target surgical location;
exposing a distal tip of the plunger beyond the distal tip of the nozzle; and
delivering material from the plunger to the target surgical location through a side port in the distal tip of the plunger.

15. The method of claim 14, wherein the percutaneous path comprises a lumen of a cannula,
wherein placing the nozzle in the percutaneous path comprises extending the distal tip of the nozzle beyond a distal tip of the cannula, and
wherein exposing the distal tip of the plunger comprises at least partially retracting the nozzle into the cannula.

16. The method of claim 14, wherein the material in the nozzle and the material from the plunger are both bone filler materials.

17. The method of claim 16, wherein the material in the nozzle and the material from the plunger are different materials.

18. The method of claim 14, wherein the target surgical location comprises a vertebra,
wherein the method further comprises creating a cavity in cancellous bone within the vertebra prior to placing the nozzle in the percutaneous path.

19. The method of claim 14, wherein the target surgical location comprises a spinal disc,
wherein the method further comprises creating a cavity in nucleus material in the spinal disc prior to placing the nozzle in the percutaneous path.

20. The method of claim 14, wherein delivering material from the plunger to the target surgical location comprises rotating the plunger within the nozzle.
